(19) United States
(12) Patent Application Publication
(10) Pub. No.: US 2002/0015716 A1
(43) Pub. Date: Feb. 7, 2002

(54) NOVEL SKIN DISINFECTION PROCEDURES

(76) Inventors: HANUMAN B. JAMPANI, GRAPEVINE, TX (US); THOMAS F. HOLLY, ARLINGTON, TX (US); JERRY L. NEWMAN, ARLINGTON, TX (US)

Correspondence Address: AUDLEY A CIAMPORCERO JR, ONE JOHNSON & JOHNSON PLAZA, NEW BRUNSWICK, NJ, 08933

(21) Appl. No.: 09/460,013

(22) Filed: Dec. 13, 1999

(51) Int. Cl. A61K 7/00

(52) U.S. Cl. 424/401

(57) ABSTRACT

Two novel skin disinfecting methods are disclosed which provide superior deposition of antimicrobial and skin-conditioning agents compared to conventional surgical scrub procedures. The novel methods are demonstrated to provide superior immediate and residual antimicrobial activity compared with the conventional surgical scrub procedures.

This application is related to U.S. patent applications, Ser. No.09/ ______, entitled DEEP PENETRATING ANTIMICROBIAL COMPOSITIONS (Attorney Doc. No. JJM-510); Ser. No. 09/ ______, entitled STABILIZED ANTIMICROBIAL SYSTEMS AND METHODS OF MAKING THE SAME (Attorney Doc. No. JJM-512); and Ser. No. 09/______, entitled THERAPEUTIC ANTIMICROBIALS COMPOSITIONS (Attorney Doc. No. JJM-513), all concurrently filed herewith and which are assigned to assignee of the present invention and incorporated by reference as if fully set forth herein.

### BACKGROUND OF THE INVENTION

1. Field of the Invention

This invention is concerned with novel skin disinfection procedures based on surgical science-proven principles for deposition of antimicrobial and skin-conditioning agents that offer superior immediate and residual antimicrobial activity compared to conventional surgical scrub procedures.

2. Related Art

Hospital acquired infections are a major concern, especially now with the increasing prevalence of antibiotic-resistant pathogenic bacteria. Healthcare personnel can transfer disease from patient to patient if handwashing protocols are not adhered to and this represents the primary manner pathogenic infections are spread in the hospital. Unfortunately, many studies have shown compliance with hygienic handwashing protocols to be seriously inadequate. In the operating room, this may take the form of fewer scrubs during the day after the initial morning scrub. Due to the nature of scrubs currently on the market, surgeons and operating room (OR) staff may compromise scrubbing frequency because of the potential for irritation. The patient is especially vulnerable to infection during invasive surgery. Because the patient's health and bacterial flora (both exogenous and endogenous) can be factors in becoming infected, all possible measures should be taken to cleanse the surgical area in pre-operative preparation. Naturally, all possible care is taken in the modern operating room to prevent airborne contamination as well (i.e., by wearing surgical masks, minimizing conversation). Another primary route of infection is contact contamination. In addition to wearing sterile surgical gloves (frequently double-layered) surgeons since the turn of the century have undergone an elaborate scrubbing ritual. The purpose of surgical scrubbing is reducing the microbial count of the skin flora to a minimum level very rapidly and keeping it down throughout a possibly lengthy operation. Around the turn of the century, surgeons began to scrub vigorously with a brush for approximately 10 minutes, and some adhere to this practice to this day. However, there is much evidence that a much shorter scrub time gives equally efficacious results, while sparing the surgeon's hands this harsh treatment. The common length of surgical scrubbing currently is 5-6 minutes.

If a surgeon acquires dermatitis or damaged skin, skin bacterial flora can actually increase. During washing or scrubbing with any soap, especially with a brush, the mechanical effect can cause shedding of skin squames. These are frequently heavily colonized by bacteria and removing them exposes new surfaces for rapid colonization. This potential for rapid recolonization is the reason plain soap is unacceptable for a surgical scrub, even though the transient flora may effectively be removed. A chemical residue is necessary for persistent activity. This is critical given the moist environment under a latex surgical glove during surgery. It is also common for blood and other contaminants to enter through pinholes in the fingertip region. Commonly used antimicrobial ingredients such as chlorhexidine gluconate (CHG) in surgical scrubs leave a chemical shield or residue against this recontamination or regrowth of resident flora.

Realizing the best defense against skin infections is healthy, intact skin surgeons strive to keep their hands supple. Unfortunately, lengthy scrubbing, especially with the harsh povidone-iodine still popularly used along with a scrub brush, can cause damage and cracking to surgeons' hands with routine use.

Scrubbing and handwashing protocols vary from hospital to hospital. The Center for Disease Control (CDC) has issued guidelines for handwashing in CDC Guidelines for Handwashing and Hospital Environmental Control, 1985. Infect. Control, 7 (4), 231-243, 1986.

The current tentative monograph (21CFR part 333.465 vol. 59, No. 116) lists the following procedure to be used in labeling all surgical scrubs:

The only variations allowed to this labeling are the scrub times which must be established by specific efficacy tests.

However, in the case of a brushless surgical wash the “device”, i.e. the scrub brush, could be omitted.

Scrub times are determined by each particular product's efficacy profile.

For products used with water:

“Clean under nails with a nail pick. Nails should be maintained with a 1 millimeter free edge. Wet hands and forearms. Apply 5 milliliters (teaspoonful) or palmful to hands and forearms. Scrub thoroughly for (3 minutes) with a sterile (scrub brush) paying particular attention to the nails, cuticles, and interdigital spaces. Rinse and repeat scrub (additional instructions for second scrub).”

For products used without water:

“Clean under nails with a nail pick. Nails should be maintained with a 1 millimeter free edge. Wet hands and forearms. Place a ‘palmful’ (5 grams) of product in one hand. Spread on both hands, paying particular attention to the nails, cuticles, and interdigital spaces, and rub into skin until dry (approximately 1 to 2 minutes). Place a smaller amount (2.5 grams) into one hand, spread over both hands to wrist, and rub into the skin until dry (approximately 30 seconds).”

The labeling of Triseptin®, the only true “brushless scrub” currently being marketed, reads:

“Directions: (1) Clean under nails with a nail pick. Nails should be maintained with a 1 millimeter free edge. (2) Wet hands and forearms. Place a palmful of Triseptin in one hand. Spread on both hands and forearms paying particular attention to the nails, cuticles, and interdigital spaces and rub into the skin for 1 minute 30 seconds. Rinse. Repeat (2).”

However, there are one or more shortcomings of current scrub methods such as:

Time: approximately 6 minutes of scrub time leads to increased healthcare costs;

Harshness: stripping action removes lipids; and does not enhance deposition;

Irritation: scrubbing with a brush causes irritations;

Cost: additional cost towards brush; and

Low Compliance: due to above disadvantages, few surgeons/staff members follow protocols exactly, possibly leading to increased post-operative complications.

Therefore, there is a need in the art for more brushless surgical handwash procedures that overcome the short-comings of conventional surgical scrub procedures and that provide a less costly and less time consuming procedure yet efficacious method such as herinafter described and claimed.

### BRIEF DESCRIPTION OF THE DRAWINGS

FIG. 1 depicts the mean log₁₀ bacteria reduction at 0 hours for the novel skin disinfection procedures of this invention (Procedures 1 and 2) compared with conventional skin disinfection procedures of the prior art (Procedures 3 and 4).

FIG. 2 depicts the mean log₁₀ bacteria reduction at 6 hours for the novel skin disinfection procedures of this invention (Procedures 1 and 2) compared with conventional skin disinfection procedures of the prior art (Procedures 3 and 4).

FIGS. 3(a) to 3(f) depict the particular hand rubbing technique used in all skin disinfection procedure evaluations (i.e., Procedures 1 to 4) described hereinafter.

### SUMMARY OF THE INVENTION

This invention relates to methods of disinfection of the skin.

One embodiment relates to a method for disinfecting skin comprising the steps of:

(a) contacting dry skin with a first application of an antimicrobial wash product and rubbing the product into the skin until the skin is dry;

(b) contacting the skin of step (a) with a second application of an antimicrobial wash product and rubbing the product into the skin until the skin is dry;

(c) contacting the skin of step (b) with water to create a lather and rubbing the lather into the skin; and

(d) contacting the skin of step (c) with water to rinse off the lather.

Another embodiment relates to a method for disinfecting skin comprising the steps of:

(a) contacting dry skin with a first application of an antimicrobial wash product and rubbing the product into the skin;

(b) wetting the skin of step (a) with water to form a lather and rubbing the lather into the skin;

(c) rinsing the skin of step (b) with water;

(d) contacting the skin of step (c) with a second application of an antimicrobial wash product and rubbing the product into the skin;

(e) wetting the skin of step (d) with water to form a lather and rubbing the lather into the skin; and

(f) rinsing the skin of step (e) with water.

Among the advantages and applications of the methods of this invention are a superior disinfection method for both alcohol- and aqueous-based formulations, substantial reductions in surgical wash time (50%) compared to some conventional methods, use as a brushless technology that does not compromise antimicrobial properties, superior deposition of antimicrobials and skin conditioning (protectant) agents than other available methods, potential use for better deposition of other therapeutically active agents such as analgesics, anti-cancer, anti-dandruff agents, anti-acne agents, anti-inflammatory, wound healing and cleansing agents, and superior residual antimicrobial properties for surgical site/catheter injection site preparation.

### DETAILED DESCRIPTION OF PREFERRED EMBODIMENTS OF THE INVENTION

The following four (4) skin disinfecting procedures were evaluated. Procedures 1 and 2 represent the novel wash procedures of this invention that are demonstrated to be far superior than in conventional procedures (i.e., Procedures 3 and 4). Note that while evaluating in the testing laboratory, clinical staff must disinfect to prevent any types of cross contamination.

Scrub Procedure One [Dry application, rub, dry application, rub, wet, lather, rinse]

Step 1.1: Volunteers' fingernails are checked to determined if they are <1.0 mm free edge. If not, they are clipped. Remove all jewelry from hands and arms.

Step 1.2: Subjects wet their hands including two-thirds of forearms under running tap water 40±2° C. for 30 seconds. Clean under fingernails and around the cuticle area with a nail cleaner. Rinse fingernails, cuticles, and hands (Skin hydration step).

Step 1.3: Subjects dry hands thoroughly with paper towels.

Step 1.4: Dispense into the subject's hands 5 ml of the assigned test article. Subjects are to distribute the material over all surfaces of the hands and lower two-thirds of the forearms taking care not to lose the substance.

Step 1.5: The material is vigorously rubbed over the hands and lower two-thirds of the forearms. Particular attention is paid to the nails, cuticles and interdigital spaces. Note: This step is performed over a period of approximately one minute. (Depositing and skin conditioning step).

Step 1.6: Dispense a second 5 ml aliquot of the test article in the subject's cupped hands. Subjects are to distribute it over all the surfaces of the hands and lower one-third of the forearm, taking care not to lose the substance.

Step 1.7: Repeat the treatment procedure described in step 1.5 except limit the scrub to the hands and lower one-third of the forearms. (An additional one minute of rubbing time) (Depositing and skin conditioning).

Step 1.8: Subjects wet hands under tap by passing hands one or two times through water.

Step 1.9: The test article is vigorously rubbed over the hands and lower one-third of the forearms paying particular attention to the finger nail region. Note: this lathering step is performed over a period of one minute (Lathering step).

Step 1.10: Rinse each hand and forearm separately for one minute per hand and shake to remove excess water (Rinsing step).

Step 1.11: Proceed with glove donning and sampling.

Total Rubbing/Lathering time: 3 minutes.

Scrub Procedure Two [Dry application, rub, wet, lather, rinse, apply to wet hands, rub' wet, lather, rinse]:

Step 2.1: Volunteers' fingernails are checked to determined if they are <1.0 mm free edge. If not, they are clipped. Remove all jewelry from hands and arms.

Step 2.2: Subjects wet their hands including two-thirds of forearms under running tap water 40±20° C. for 30 seconds. Clean under fingernails and around cuticle area with a nail cleaner. Rinse fingernails and hands (Skin hydration step).

Step 2.3: Subjects dry hands thoroughly with paper towels.

Step 2.4: Dispense into the subject's hands 5 ml of the assigned test article. Subjects are to distribute the material over all surfaces of the hands and lower two-thirds of the forearms taking care not to lose the substance.

Step 2.5: The material is vigorously rubbed over the hands and lower two-thirds of the forearms. Particular attention is paid to the nails and interdigital spaces.

Note: This step is performed over a period of approximately 45 seconds. (Depositing and skin conditioning)

Step 2.6: Subjects wet hands under tap by passing hands one or two times through water. (Rinsing step)

Step 2.7: The test article is vigorously rubbed over the hands and lower one-third of the forearms paying particular attention to the fingernail and cuticle region. Note: this lathering step is to be performed over a period of 45 seconds. (Lathering step)

Step 2.8: Subjects rinse both hands and the lower one-third of the forearms for 30 seconds under running tap water 40±2° C. and shake to remove excess water. (Rinsing step)

Step 2.9: Repeat steps 2.4 through 2.7 limiting application and washing to hands and lower one-third of the forearms. Note: Subjects hands will be wet when the assigned test article is applied this time. As before rub for 45 seconds and lather for 45 seconds.

Step 2.10: Rinse each hand and forearm separately for one minute per hand.

Step 2.11: Don gloves and proceed with sampling.

Total Rubbing/Lathering Time: 3.0 minutes.

Scrub Procedure Three [Apply to wet hands, rub, reapply, rub, rinse]:

Step 3.1: Volunteers' fingernails are checked to determine if they are <1.0 mm free edge. If not, they are clipped. Remove all jewelry from hands and arms.

Step 3.2: Subjects wet their hands including two-thirds of forearms under running tap water 40±2° C. for 30 seconds. Clean under fingernails, and around cuticle area with a nail cleaner. Rinse fingernails, cuticles, and hands.

Step 3.3: Subjects shake excess water from hands.

Step 3.4: Dispense into the subject's hands 5 ml of the assigned test article. Subjects are to distribute two-thirds of the forearms taking care not to lose the substance.

Note: Product is applied to moist hands in this step.

Step 3.5: The material is vigorously rubbed over the hands and lower two-thirds of the forearms. Particular attention is paid to the nails, cuticles, and interdigital spaces. Note: This step is performed over a period of approximately 1 minute.

Step 3.6: Repeat steps 3.4 through 3.5 limiting application to hands and lower one-third of the forearms. Material will be added to hands, which have been rubbed. The 2nd application is also followed by a 1 minute rub.

Step 3.7: Subjects wet hands under tap by passing hands one or two times through water.

Step 3.8: The test article is vigorously rubbed over the hands and lower one-third of the forearms paying particular attention to the fingernail and cuticle region.

Note: This lathering step is performed over a period of one minute.

Step 3.9: Rinse each hand and forearm separately for one minute per hand.

Step 3.10: Don gloves and proceed with sampling.

Total Rubbing/Lathering Time: 3.0 minutes.

Scrub Procedure Four [Apply to wet hands, lather, rinse, reapply to wet hands, lather, rinse]:

Step 4.1: Volunteers' fingernails are checked to determined if they are <1.0 mm free edge. If not, they are clipped. Remove all jewelry from hands and arms.

Step 4.2: Subjects wet their hands including two-thirds of forearms under running tap water 40±2° C. for 30 seconds. Clean under fingernails and around cuticle area with a nail cleaner. Rinse fingernails and hands.

Step 4.3: Shake excess water from hands.

Step 4.4: Dispense into the subject's hands 5 ml of the assigned test article. Subjects are to distribute the material over all surfaces of the hands and lower two-thirds of the forearms taking care not to lose the substance.

Note: Product is applied to moist hands in this step.

Step 4.5: The material is vigorously rubbed over the hands and lower two-thirds of the forearms. Particular attention is paid to the nails, cuticles, and interdigital spaces.

Note: This step is performed over a period of approximately 45 seconds.

Step 4.6: Subjects wet hands under tap by passing hands one or two times through water.

Step 4.7: The test article is vigorously rubbed over the hands and lower one-third of the forearms paying particular attention to the finger nail region. Note: this lathering step is to be performed over a period of 45 seconds.

Step 4.8: Subjects rinse both hands and the lower one-third of the forearms for 30 seconds under running tap water 40±2° C. and shake excess water from hands.

Step 4.9: Repeat steps 4.4 through 4.7 limiting application and washing to hands and lower one-third of the forearms. Material will be added to wet hands, as before, followed by a 45 second rub, re-wetting, and a 45 second lathering step.

Step 4.10: Rinse each hand and forearm separately for one minute per hand.

Step 4.11: Don gloves and proceed with sampling.

Total Rubbing/Lathering Time: Approximately 3.0 minutes.

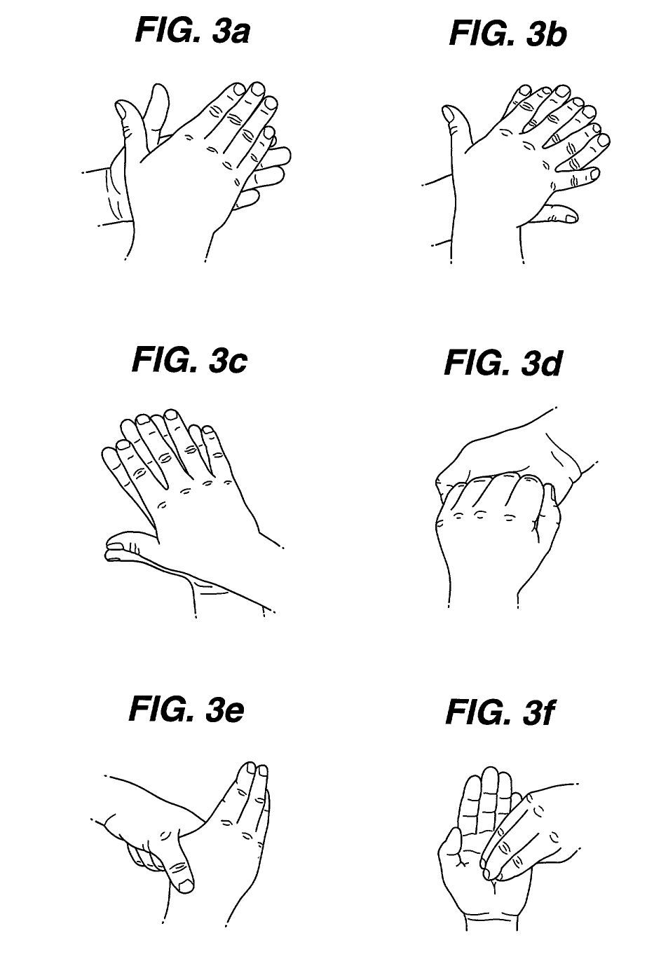

The basic technique used to contact the skin with the antimicrobial solutions used in Procedures 1 to 4 was in general the same for all Procedures as illustrated in FIGS. 3(a) to 3(f). In particular, FIG. 3(a) depicts palm to palm rubbing; 3(b) depicts right palm over left dorsum and left palm over right dorsum rubbing; 3(c) depicts palm to palm interlaced rubbing; 3(d) depicts rubbing with backs of fingers to opposing palms with fingers interlocked; 3(e) depicts rotational rubbing of the right thumb clasped in the left palm, this step is repeated vice versa with rubbing of the left thumb in the right palm; and 3(f) depicts rotational rubbing backwards and forwards with clasped fingers of right hand in left palm, this step is repeated vice versa for the left hand in the right palm.

The antimicrobial composition used for Procedures 1 to 4 where the same of all Procedures and was of the following composition as indicated in Table 1. Note all percentages are based on weight percent of the composition unless otherwise noted.

TABLE 1
ANTIMICROBIAL COMPOSITION

| Component | Weight % |
|---|---|
| Ethanol | 62.25 |
| | (70% V/V) |
| Glycerin | 5.0 |
| Hydroxypropyl Cellulose | 1.0 |
| Isolene | 1.0 |
| Silsoft PEDM¹ | 0.05 |
| Mearlmaid OL² | 0.1 |
| D.I. Water | 12.74 |
| Propylene Glycol | 5.0 |
| Cetrimonium Chloride (29%) | 0.86 |
| Phospholipid CDM³ | 1.5 |
| Benzalkonium Chloride (50%) | 0.2 |
| Phenoxyethanol | 1.0 |
| Benzethonium Chloride | 0.1 |
| Cetylpyridinium Chloride | 0.1 |
| Dowicil 200⁴ | 0.25 |
| Plantaren 2000⁵ | 3.6 |
| Mackam CBS 50G⁶ | 2.4 |
| PPG-40 Diethylmonium Chloride⁷ | 1.2 |
| Fragrance⁸ | 0.15 |
| Lambent Quat AD⁹ | 0.5 |
| Glyceryl Laurate | 1.5 |

Notes:
¹phenylethyl dimethicone, Witco Corp., Osi Specialties, Inc.
²isopropyl alcohol (and) guanine (and) polysorbate-80 (and) methylparaben (and) propylparaben, Engelhard Corp
³coco (PG) dimonium chloride phosphate, Mona Industries, Inc.
⁴quaternium 15, Dow Chemical
⁵decyl polyglucose (polyalkylglycoside) (Henkel)
⁶cocamidopropyl hydroxysultaine (50%), McIntyre
⁷Emcol CC-42, Witco Corp., Osi Specialties, Inc.
⁸Seafoam GGE, Firmenich
⁹silicone quaternary compound, Lambent Technology

The effectiveness of each washing procedure is reducing the bacterial population of the treated skin, both immediately (0 hours) and after 6 hours were determined by measuring the log₁₀ reduction in bacterial counts.

This test method is conducted on subjects selected from a group of volunteers who have refrained from using any antimicrobials for at least two weeks prior to initiation of the test. Sufficient number of subjects are selected from this group on the basis of high initial bacteria count, 1×10⁵ per hand as determined by baseline measurements of the bacteria on their hands.

The selected subjects perform a simulated surgical handwash under the supervision of an individual competent in aseptic technique. One hand is sampled after the surgical handwash and the other hand after 6 hours. The difference between the base line and the recovered organisms after surgical hand wash gives the antimicrobial effectiveness of test formulations. Also it should be noted that as used herein and as claimed the term “dry”, is intended to also have the meaning of “substantially” or “nearly” dry as in practice it may not be practical or possible to achieve a completely “dry” application of the wash product as used in method of the invention. Also the precise composition method of the particular handwash will have an effect on the degree and speed of drying of the handwash. Desirably, use of a handwash composition having from about 30 to about 90 (V/V)% alcohol will aid in shortening the time for drying.

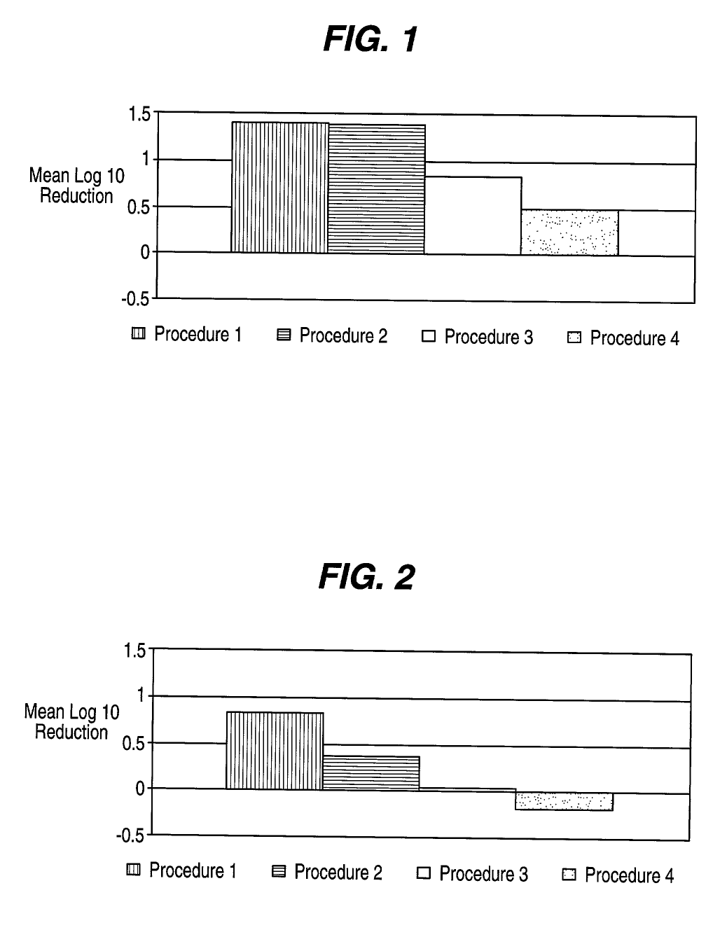

The results are presented in Table 2 below and graphically in FIGS. 1 and 2.

TABLE TWO

| | | Mean Log₁₀ |
|---|---|---|
| Surgical Handwash Procedure | | Reductions |
| Procedure One | 0 Hours | 1.41 |
| | 6 Hours | 0.84 |
| Procedure Two | 0 Hours | 1.39 |
| | 6 Hours | 0.36 |
| Procedure Three | 0 Hours | 0.84 |
| | 6 Hours | 0.02 |
| Procedure Four | 0 Hours | 0.47 |
| | 6 Hours | −0.2 |

It is clear from Table 2 and FIGS. 1 and 2 that Procedures 1 and 2 showed greater than 1 log₁₀ reduction at zero time, showing clinical significance, whereas Procedures 3 and 4 did not. Looking at the combination of 0 and 6 hour results, Procedure 1 is clearly superior. Though the 0 hour reductions for Procedure 1 and 2 are not significantly different, the residual efficacy using method one is significantly improved. Not wishing to be bound by any particular theory, Applicants believe that the results clearly suggest that the thickeners and other skin conditioning agents form a reservoir to hold antimicrobials to offer slow release for residual activity. Also, this has marked improvements in the skin conditioning feel due to retention of moisture offering molecules in the stratum corneum. This demonstrates the improved result one can expect from the double-dry application and the increased penetrating ability of the formula using this method.

Once again, in addition to enhanced efficacy, especially residual activity, of Procedure 1, the penetration of conditioners is also enhanced. Surgeons can expect to have better skin integrity, and a lesser incidence of dermatitis, and improved antimicrobial efficacy when using this method. In combination with the added residual activity, the better skin condition will give additional protection to the surgeon in case of accidental glove punctures. In addition this procedure has the following advantages to surgeons or OR staff members:

Easy application of the product

Elimination of scrub brush

Mild surgical hand washing vs. harsh surgical scrubbing

50% reduction in surgical handwashing time

There are many other applications of this handwashing procedure. In addition to surgical applications, it could be used in a healthcare personnel handwash application or many other handwashing and cleansing situations particularly in non-health care settings. The depositing of different types of active ingredients as well as conditioners is enhanced with this procedure.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A method for disinfecting skin comprising the steps of:
(a) contacting dry skin with a first application of an antimicrobial wash product and rubbing the product into the skin until the skin is dry or substantially dry;
(b) contacting the skin of step (a) with a second application of an antimicrobial wash product and rubbing the product into the skin until the skin is dry or substantially dry;
(c) contacting the skin of step (b) with water to create a lather and rubbing the lather into the skin; and
(d) contacting the skin of step (c) with water to rinse off the lather.

2. The method of claim 1, further comprising the step of rinsing the skin with water and then thoroughly drying the skin before step (a).

3. The method of claim 1, wherein the skin to be treated is the skin of the hands or forearms and that prior to step (a), the method further comprising the step of wetting the skin of the hands or forearms with water, cleaning under the fingernails and around the cuticle area of the hands with a nail cleaner, rinsing the fingernails and hands with the water, and thoroughly drying the wetted skin areas.

4. The method of claims 1 to 3, wherein the water used is tap water available at a temperature of about 40±2° C.

5. The method of claim 3, wherein the water used is tap water available at a temperature of about 40±2° C., and the rubbing parts of steps (a), (b) and (c) are performed over a period of approximately 3 minutes.

6. A method for disinfecting skin comprising the steps of:
(a) contacting dry skin with a first application of an antimicrobial wash product and rubbing the product into the skin;
(b) wetting the skin of step (a) with water to form a lather and rubbing the lather into the skin;
(c) rinsing the skin of step (b) with water;
(d) contacting the skin of step (c) with a second application of an antimicrobial wash product and rubbing the product into the skin;
(e) wetting the skin of step (d) with water to form a lather and rubbing the lather into the skin; and
(f) rinsing the skin of step (e) with water.

7. The method of claim 6, further comprising the step of rinsing the skin with water and then thoroughly drying the skin before step (a).

8. The method of claim 6, wherein the skin to be treated is the skin of the hands or forearms and that prior to step (a), the method further comprising the step of wetting the skin of the hands or forearms with water, cleaning under the fingernails, and around cuticle area of the hands with a nail cleaner, rinsing the fingernails and hands with water and thoroughly drying the wetted skin areas.

9. The method of claims 6 to 8, wherein the water used is tap water available at a temperature of about 40±2° C.

10. The method of claim 8, wherein the water used is tap water available at a temperature of about 40±2° C., and the rubbing part of steps (a) and (d) are each done for a duration of about 45 seconds and the rubbing and lathering parts of steps (b) and (e) are each done for a duration of about 45 seconds, and in total 3 minutes.

11. The method of claim 1 or 6 wherein the method is used for deposition of therapeutically active agents.

12. The method of claim 11, wherein the active agents are selected from antimicrobials, anti-inflammatory compounds, anticancer compounds, skin protectants, analgesics, anti-dandruff agents, anti-acne agents, and wound healing and wound cleaning agents.

13. The method of claim 1 or 6 wherein the method is used for surgical site or catheter site preparation.

14. The method of claim 1 or 6, wherein the rubbing or lathering steps are used with or without a brush.

15. The method of claim 1 or 6, wherein the antimicrobial wash products comprise from about 30 to about 90 (V/V)% of an alcohol.
